(19) United States
(12) Patent Application Publication
(10) Pub. No.: US 2015/0320790 A1
(43) Pub. Date: Nov. 12, 2015

(54) Antacid

(71) Applicant: Tummy Company, Inc., Phoenix, AZ (US)

(72) Inventor: Doug daCosta, Paradise Valley, AZ (US)

(73) Assignee: Tummy Company, Inc., Phoenix, AZ (US)

(21) Appl. No.: 14/120,235

(22) Filed: May 9, 2014

(51) Int. Cl. A61K 33/00; A61K 33/10

(52) U.S. Cl. CPC A61K 33/00; A61K 33/10

(57) ABSTRACT

A low-viscosity, high pH antacid composition comprising: sodium bicarbonate, potassium bicarbonate; calcium carbonate, sweetener, and water. The concentration of sodium bicarbonate in the composition of the invention is preferably in the range of from 1% to 3% by weight. The concentration of potassium bicarbonate in the composition of the invention is preferably in the range of from 0.5% to 1% by weight. The concentration of calcium carbonate in the composition of the invention is preferably in the range of from 0.25% to 1% by weight. The concentration of sweetener in the composition of the invention is preferably in the range of from 0.02% to 0.04% by weight. The remainder of the composition is a low viscosity transport agent, such as water. Additionally, stabilizers may be added and flavoring/coloring agents may be added.

### FIELD OF THE INVENTION

This invention relates to compositions and methods for delivering antacids to user. More particularly, the invention relates to a method and composition for delivering a low-viscosity, high pH antacid to a user.

### BACKGROUND OF THE INVENTION

Acid indigestion, or heartburn, is one of the most frequently occurring human malaise and is responsible for substantial physical discomfort and economic loss. Smoking and increased body mass index were associated with heartburn occurrence. Heartburn sufferers reported more atypical gastro-esophageal reflux disease symptoms, e.g. chest pains, dysphagia and globus. They were more likely to consult physicians, and have an increased frequency and number of days of absenteeism, irrespective of upper gastrointestinal or non-fastrointestinal-related illnesses. They also experiences sleep disturbances more frequently.

Thus, there is a present and on-going need for improved heartburn therapies.

### SUMMARY OF THE INVENTION

In light of the foregoing, therefore, it is a first aspect of the invention to provide a novel antacid composition.

It is a further aspect of this invention to provide a novel antacid composition with a viscosity between 0.89 cP at about 25° C. and 25 cP at about 25° C.

It is a still further aspect of this invention to provide a novel antacid composition with a static pH around 7.75 to 9.5 and a viscosity between 0.89 cP at about 25° C. and 25 cP at about 25° C.

It is a still further aspect of this invention to provide a novel antacid composition with a static pH around 7.75 to 9.5.

It is a still further aspect of this invention to provide a novel antacid container having substantially transparent composition viewing window located in a side of the antacid container.

The novel features that are considered characteristic of the invention are set forth with particularity in the appended claims. The invention itself, however, both as to its structure and its operation together with the additional object and advantages thereof will best be understood from the following description of the preferred embodiment of the present invention when read in conjunction with the accompanying drawings. Unless specifically noted, it is intended that the words and phrases in the specification and claims be given the ordinary and accustomed meaning to those of ordinary skill in the applicable art or arts. If any other meaning is intended, the specification will specifically state that a special meaning is being applied to a word or phrase. Likewise, the use of the words “function” or “means” in the Description of Preferred Embodiments is not intended to indicate a desire to invoke the special provision of 35 U.S.C. §112, paragraph 6 to define the invention. To the contrary, if the provisions of 35 U.S.C. §112, paragraph 6, are sought to be invoked to define the invention(s), the claims will specifically state the phrases “means for” or “step for” and a function, without also reciting in such phrases any structure, material, or act in support of the function. Even when the claims recite a “means for” or “step for” performing a function, if they also recite any structure, material or acts in support of that means of step, then the intention is not to invoke the provisions of 35 U.S.C. §112, paragraph 6. Moreover, even if the provisions of 35 U.S.C. §112, paragraph 6, are invoked to define the inventions, it is intended that the inventions not be limited only to the specific structure, material or acts that are described in the preferred embodiments, but in addition, include any and all structures, materials or acts that perform the claimed function, along with any and all known or later-developed equivalent structures, materials or acts for performing the claimed function.

### BRIEF DESCRIPTION OF THE FIGURES

The invention will be more fully understood by referring to the following Detailed Description of Specific Embodiments in conjunction with the Drawings, of which:

FIG. 1 is a view of a container label having a composition viewing window located within the circumference of the container label.

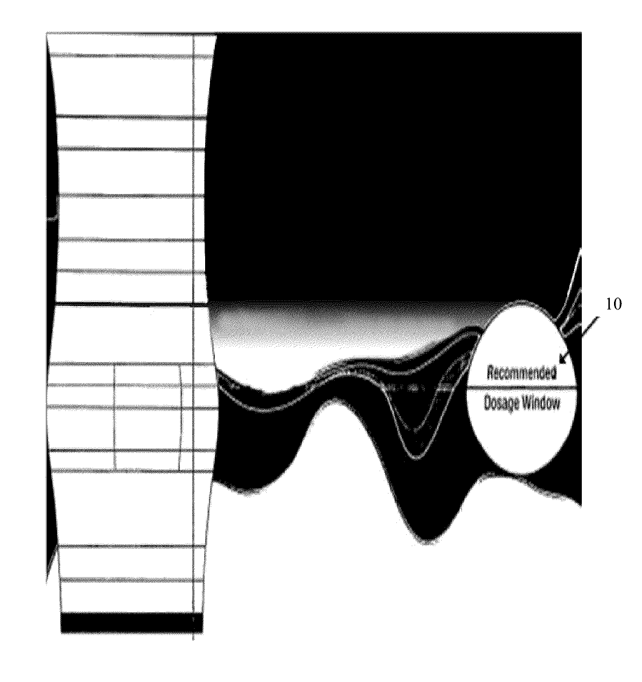

### DETAILED DESCRIPTION OF THE INVENTION

The present invention is a novel antacid composition useful for treating acid reflux, heart burn and like problems. The instant composition is a low viscosity and/or high pH composition that aid in quick treatment of the acid associated problem.

It has been shown that problems that beneficially respond to antacid therapy do so in direct proportion to the amount and pH of antacid ingested and to its duration of action within the stomach. Prior liquid antacids have heretofore required stabilization which has been achieved by the addition of viscosity increasing additives. In such systems there is a low limit of antacid concentrations attainable beyond which an undesirable thickened or paste consistency occurs. As a result, there is a high concentration of non-active ingredients in the suspension. Other means of suspension used in the prior art include formation of supersized floes and chemical gel formation, all of which produce undesirable, unpalatable antacid compositions and greatly limit the antacid concentration attainable.

Furthermore, because of the relatively high concentration of the non-active ingredients added to achieve stability of these suspensions of the prior art, there is interference of interaction between the active antacid and the gastro-intestinal secretions and/or diseased areas. This results in delayed on-set of action as well as preventing part of the ingested antacid from ever interacting with the gastric acid before being washed out of the stomach. This delayed on-set of action, caused by interference of interaction of the active antacid, also prevents direct action on the irritated, inflamed or ulcerated portions of the lower esophagus through which the products of the prior art rapidly pass without desirable active interactions.

What beneficial effects that are attained in the lower esophagus by the prior art is only that of reduced gastric acidity which secondarily reduces the irritation to the lower esophagus produced by reflux of gastric contents into this anatomical area.

In addition, the stability of these suspensions has been difficult to maintain during long shelf life. These products of the prior art have varying degrees of stability and shelf life wherein prolonged standing without agitation results in undue settling, caking and partial gelation, which require considerable effort and agitation to bring about thorough resuspension, if totally resuspendable at all. Also, should these products, because of a loose bottle cap, be allowed to dry out or partially dry out, addition of water will not bring about a resuspension of the ingredients without the use of techniques or equipment not commonly available to the pharmacist or patient user.

The prior liquid products, because of their low concentration of antacid ingredients, must be consumed in large volumes to be efficacious and furthermore, require frequently repeated dosages. In attempting to achieve prolongation of therapeutic action, the very viscous antacid products of the prior art have been combined with drugs to slow down the emptying rate of the stomach. These additional additive products have undesirable side effects. Many liquid products now commercially available have been described in advertising and clinical reports as possessing increased viscosity as a desirable characteristic to aid in prolonging antacid activity by increased adhesion and decreased flowability in the upper gastro.-intestinal tract. However, when these products, although of increased viscosity when ingested, mix with the secretions of the stomach, there can only be a dilution effect of the thickening agents or breakdown of the chemical gel with resulting decreased viscosity of the suspension within the stomach, and thus, greater fluidity allowing more rapid emptying from the stomach. In addition these products are all of low range concentration of the active antacid ingredient and, therefore, are subject to the disadvantages previously described.

The literature is full of studies directed to attempting to improve upon the problems of high volume, frequently repeated dosages in antacid therapy by means of the methods mentioned above and by constantly seeking different chemical active ingredient forms. All have failed appreciably to solve these major problems without undue side effects from additives used or by greatly reduced palatability and decreased patient acceptance.

The solid antacid products of the prior art have also failed to attain the desired efficaciousness as described above, because their physical form and chemical ingredients require that either they be swallowed in solid form or be dissolved in large volumes of orally generated saliva. Also, the relatively small amount of active antacid ingredient per tablet requires ingestion of many tablets and frequently repeated doses to achieve desirable therapeutic results. In any case, once these antacid ingredients enter the stomach they face the same disadvantages as described above for liquid antacid products. In addition, because of the gritty, dry and bland taste and unpalatable oral feel, these products also are met with a low level of patient acceptance.

### DETAILED DESCRIPTION OF SPECIFIC EMBODIMENTS

The present invention is a novel antacid composition useful for treating acid reflux, heart burn and like problems. The instant composition is a low viscosity and/or high pH composition that aid in quick treatment of the acid associated problem.

The instant composition has a viscosity in the range of 0.89 to 25 centipoise, preferably 5 to 15 centipoise. The viscosity of the composition is important because it facilitates transport of the composition to the stomach. When the viscosity is greater than about 25 centipoise, the composition tends to coat the esophagus instead of being delivered directly to the stomach cavity.

The low viscosity composition of the instant invention utilizes a combination of sodium and potassium carbonate as the active. This greatly reduces the amount of sodium supplied by a single dose of the instant composition. As noted, composition viscosities in excess of about 25 centipoise are believed to interfere with the transport of the composition to the stomach.

Viscosity measurements recited herein were obtained using the Brookfield SynchoLectric Viscometer for the measurement of the apparent Viscosity of Newtonian and NonNewtonian materials at low shear rates at given rotational speeds (ASTM D1824-87). See also ASTM D 1084-88, ASTM D2196-86 and other ASTM protocols concerning the measurement of viscosity.

In another embodiment of the invention, we provide a ready to consume, liquid, antacid composition that has a stable pH ranging from 7.75 to 9.5. This is important since prior art antacids are either in a tablet form or only have a pH less than 7.75. Additionally, a greater pH allows for a lower dose to neutralize an equivalent amount of acid.

In a further embodiment of the invention, we provide an improved container for delivering a low-viscosity antacid. This improved container comprises a container with a bottom, a circumferential sidewall, an open top and a cap to seal the open top. The improvement in the container further comprises a container label 1 that has a top side, a bottom side, and a circumference, further the container label has a composition viewing window 10 located inside of the circumference. In one embodiment the window 10 is completely transparent. In a second embodiment the window 10 is slightly opaque, yet transparent enough to allow viewing of the level of the contents of the container. In a third embodiment, the window 10 is an aperture in the container label.

The following examples depict the presently preferred embodiments of the invention for the purposes of illustrating the practice thereof and not be way of limitation of the scope of the invention. In the examples, all proportions are by weight, unless otherwise noted.

### Example 1

| INGREDIENT | Weight % |
|---|---|
| Sodium Bicarbonate USP | 1.75 |
| Potassium Bicarbonate USP | 0.75 |
| Calcium Carbonate | 0.435 |
| Sweetener | 0.022 |
| Flavor (Dark Cherry) | 0.2 |
| Water | 96.743 |
| pH | 8.00-8.22 |
| Viscosity | |

### Example 2

| INGREDIENT | Weight % |
|---|---|
| Sodium Bicarbonate USP | 2.735 |
| Potassium Bicarbonate USP | 0.975 |
| Calcium Carbonate | 0.500 |
| Sweetener | 0.022 |
| Flavor (Dark Cherry) | 0.4 |
| Parabens | 0.100 |
| Water | 95.676 |
| pH | 8.3-8.7 |
| Viscosity | |

### Example 3

| INGREDIENT | Weight % |
|---|---|
| Sodium Bicarbonate USP | 1.25 |
| Potassium Bicarbonate USP | 0.75 |
| Calcium Carbonate | 0.435 |
| Sweetener | 0.022 |
| Flavor (Stawberry/Lemon) | 0.350 |
| Water | 97.193 |
| pH | 8.00-8.2 |
| Viscosity | |

The above examples yield compositions having viscosities that fall between 0.89 and 25 cP, more specifically between 5 and 15 cP.

The concentration of sodium bicarbonate in the composition of the invention is preferably in the range of from 1% to 3% by weight. The concentration of potassium bicarbonate in the composition of the invention is preferably in the range of from 0.5% to 1% by weight. The concentration of calcium carbonate in the composition of the invention is preferably in the range of from 0.25% to 1% by weight. The concentration of sweetener in the composition of the invention is preferably in the range of from 0.02% to 0.04% by weight. The remainder of the composition is a low viscosity transport agent, such as water.

Stabilizers, such as one of the Parabens may be added. Flavoring/coloring agents may be added. A gelling agent may be added, but only as long as the viscosity still falls below 25 cP. Further, antioxidants may be added in order to increase stability.

The preferred embodiment of the invention is described above in the Drawings and Description of Preferred Embodiments. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s). The foregoing description of a preferred embodiment and best mode of the invention known to the applicant at the time of filing the application has been presented and is intended for the purposes of illustration and description. It is not intended to be exhaustive or to limit the invention to the precise form disclosed, and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated.

1. An antacid composition comprising:
a. sodium bicarbonate in the range of from 1% to 3% by weight,
b. potassium bicarbonate in the range of from 0.5% to 1% by weight
c. calcium carbonate in the range of from 0.25% to 1% by weight,
d. sweetener in the range of from 0.02% to 0.04% by weight, and
e. water to 100%.

2. The composition according to claim 1 wherein said antacid composition has a viscosity between about 0.89 centipoise (cP) and about 25 cP at about 25° C.

3. The composition according to claim 1 wherein said antacid composition has a viscosity between about 5 cP and about 15 cP at about 25° C.

4. The composition according to claim 1 wherein said antacid composition has a pH ranging between about 7.75 and about 9.5.

5. The composition according to claim 1 wherein said antacid composition has a pH greater than about 8.

6. The composition according to claim 1 wherein said antacid composition has a pH greater than about 8.5.

7. The composition according to claim 1 wherein said antacid composition has a pH greater than about 9.

8. The composition according to claim 1 wherein said antacid composition has a pH greater than about 9.5.

9-11. (canceled)

12. An aqueous antacid composition comprising:
a. about 1 wt % to about 3 wt % sodium bicarbonate;
b. about 0.5 wt % to about 1 wt % potassium bicarbonate; and
c. about 0.25 wt % to about 1 wt %. calcium carbonate;
wherein the antacid composition has a viscosity between about 0.89 cP and about 25 cP at about 25° C.

13. The antacid composition of claim 12, wherein the composition has a pH between about 7.75 and about 9.5.

14. The antacid composition of claim 12 further comprising about 0.02 wt % to about 0.04 wt % sweetener.

15. An aqueous antacid composition comprising an effective amount of bicarbonate selected from the group consisting of sodium, potassium and calcium bicarbonate, wherein the composition has a viscosity between about 5 cP and about 15 cP at about 25° C. and a pH above about 7.75.

16. An aqueous antacid composition comprising:
a. about 1 wt % to about 3 wt % sodium bicarbonate; and
b. about 0.5 wt % to about 1 wt % potassium bicarbonate;
wherein the antacid composition has a viscosity between about 5 cP and about 15 cP at about 25° C.

17. The antacid composition of claim 16, wherein the composition has a pH between about 7.75 and about 9.5.

18. The antacid composition of claim 16 further comprising about 0.25 wt % to about 1 wt % calcium carbonate.

19. A method of preparing an aqueous antacid composition, the method comprising combining:
a. about 1 wt % to about 3 wt % sodium bicarbonate;
b. about 0.5 wt % to about 1 wt % potassium bicarbonate; and
c. about 0.25 wt % to about 1 wt %. calcium carbonate; wherein the antacid composition has a viscosity between about 5 cP and about 15 cP at about 25° C. and a pH between about 7.75 and about 9.5.

20. A method for providing heartburn relief to a subject suffering heartburn, comprising administering an antacid composition according to claim 16 to a subject suffering heartburn, whereby said subject's heartburn is relieved.
